(19) United States
(12) Patent Application Publication
(10) Pub. No.: US 2016/0220144 A1
(43) Pub. Date: Aug. 4, 2016

(54) METHOD AND MAGNETIC RESONANCE APPARATUS FOR SETTING A PATIENT POSITION AND/OR AT LEAST ONE SLICE POSITION

(71) Applicant: Siemens Aktiengesellschaft, Muenchen (DE)

(72) Inventor: Dominik Paul, Bubenreuth (DE)

(73) Assignee: Siemens Aktiengesellschaft, Muenchen (DE)

(21) Appl. No.: 15/009,901

(22) Filed: Jan. 29, 2016

(30) Foreign Application Priority Data
Jan. 29, 2015 (DE) 102015201521.1

(51) Int. Cl. A61B 5/055; A61B 5/00; G06F 3/0488; G06F 3/0484; G06F 3/0482

(52) U.S. Cl. CPC A61B 5/0555; G06F 3/04845; G06F 3/04847; G06F 3/04842; G06F 3/0482; G06F 3/0488; A61B 5/70; A61B 5/7475; A61B 5/7435; A61B 5/7425; A61B 5/7445

(57) ABSTRACT

In a method and magnetic apparatus for setting a patient position and/or at least one slice position of a target slice to be acquired in a scanner of the apparatus, at least two slices images showing localization slices of the patient, perpendicular to one another and with a defined position with respect to the homogeneity volume of the scanner, are acquired. At least one of the slice images is displayed on a touchscreen with at least one marker line showing the position of at least one other localization slice in the slice image. Following a relative displacement, described by displacement data, of at least one of the lines and the displayed slice image, by user interaction with the touchscreen, the patient position and/or the slice position of the target slice to be acquired are adapted in accordance with the displacement data.

### BACKGROUND OF THE INVENTION

1. Field of the Invention

The invention concerns a method for setting a patient position and/or at least one slice position of a target slice to be acquired in a magnetic resonance apparatus, as well as a magnetic resonance apparatus for implementing such a method.

2. Description of the Prior Art

Magnetic resonance imaging has become an established modality in medical practice. If specific anatomical structures are of interest, the patient has to be positioned correctly in the patient receiving area of the scanner of the magnetic resonance apparatus, in particular as optimally as possible with respect to the homogeneity volume of the scanner. To enable this, it is known to provide a laser sight in the region of the center of the homogeneity volume. The user situated outside the patient receiving area uses the laser sight to set a position on the patient and then automatically move the patient to a patient position at which the marked point lies in the homogeneity volume, in particular in the region of central slices. In this case, the user is only able to view the outside of the body, so that he or she is unable to see the anatomical structures at which the imaging is targeted, and hence errors may occur in the positioning.

Also known are magnetic resonance apparatuses with a patient support, for example a patient bed, which is designed only for movement in certain directions, such as vertically. Other degrees of freedom are covered by the appropriate displacement of slices in order to be able to acquire the desired imaging target structures. In this known state of the art, this can also be performed by means of laser sights.

### SUMMARY OF THE INVENTION

An object of the invention is to provide a method for more precise positioning of patients and/or slices from which raw data are to be acquired in a magnetic resonance apparatus.

This object in accordance with the invention by a wherein at least two slice images showing localization slices, which are perpendicular to one another and have a defined position with respect to the homogeneity volume of the magnetic resonance scanner, are acquired inside the patient arranged on a patient support in the patient receiving area. At least one of the slice images is displayed on a touchscreen of the magnetic resonance apparatus with at least one marker line showing the position of at least one other slice in the slice image. Following a relative displacement described by displacement data of the marker line, or at least one marker line if there are multiple marker lines, and the slice image displayed by user interaction with the touchscreen, the patient position and/or the slice position of the target slice to be acquired are adapted in accordance with the displacement data.

The inventive method for positioning a patient and/or target slices to be acquired in a patient with a magnetic resonance apparatus uses a touchscreen, i.e. a screen which can also be used as an input device. In this case, the patient is first introduced into the patient receiving area, and a starting position of the patient can be selected dependent on the parameters of the planned image acquisition. Parameters of this kind can be, for example, information about any local coils that are connected and/or a setting in a patient registration. If, for example, head coils are connected as local coils, imaging of the head can be assumed; back coils are indicative of imaging in the region of the torso. The patient registration also frequently includes useful information about the choice of starting position, for example an imaging target and/or an anatomical structure to be acquired.

If the patient is within the patient receiving area at the starting position, at least two slice images are acquired in localization slices perpendicular to one another, preferably depicting the main planes of the body. Therefore, it is preferable that two slice images are acquired from the group including a transverse slice image, a coronal slice image and a sagittal slice image. It is particularly preferable for slice images to be acquired in coronal and transverse localization slices. Applications are conceivable in which, additionally or alternatively, a sagittal slice image, namely a slice image in a sagittal body plane, is also useful.

In an embodiment of the invention, the slice images are acquired with a localizer sequence. Preliminary localizer acquisitions are known in the art; they can be acquired extremely quickly and are therefore particularly suitable to be used for the purposes of the positioning method according to the invention.

The slice images initially acquired have a spatial or geometric relation to a specific, defined position in the homogeneity volume. Preferably ,the localization slices that are initially acquired pass through the center of the homogeneity volume, i.e. through the isocenter of the magnetic resonance scanner so that the slice selection is transparent to the user and particularly useful to the user for orientation.

User interaction with the touchscreen now enables the user to change the relative position of the at least one marker line and the slice image displayed. For example, to optimize the positioning of the patient by subsequent control of the patient support apparatus dependent on displacement data and/or the slice positioning the corresponding image acquisition parameters can be adapted by the user such that the desired imaging target can be achieved. If, for example with a displayed coronal slice image, the relative position of the slice image and the marker line relating to a transverse slice image is changed, the patient support then moves the patient in the longitudinal direction if the patient support has such an adjustment capability.

An operating component that has not previously been used in this context, namely a touchscreen, is used to provide a simple, easy-to-operate possibility for positioning. This provides extremely precise positioning since it is immediately clear from the slice images how the patient and/or the target slices are positioned.

In an embodiment of the invention, the user interaction takes the form of displacement of the marker line on the slice image, or displacement of the slice image toward the marker line. It is preferable for it to be possible to move the marker line on the slice image, which is preferably displayed in its entirety. In both cases mentioned, the user interacts directly with the slice image displayed and hence can establish a direct relationship between the anatomy and the positioning in order to manipulate the positioning directly in accordance with the user's wishes. This permits intuitive control of the magnetic resonance apparatus, which is easy to understand, learn and carry out.

Another embodiment provides an additional possibility for the user interaction, in the form of at least one operator element for the fine adjustment of the displacement to be displayed on the touchscreen, in particular superimposed on the image. For example, arrows can be displayed adjacent to the at least one marker line the actuation of which achieves an extremely small displacement, such as by one pixel. In this case, it is preferable for all operating possibilities and operator elements to be depicted as integrated in the slice image displayed or superimposed thereon so that the maximum display area of the touchscreen can be used for the at least one slice image displayed.

It is for every manipulation, namely relative displacement, of the marker line or the slice image immediately to not immediately result in an adjustment action of the magnetic resonance apparatus, at least as far as the positioning of the patient is concerned, which should also be performed in as targeted a manner as possible. Therefore, in an embodiment of the invention, at least a change to the patient position takes place only following an operator confirmation action. Such an operator confirmation action can be achieved without additional operator elements by double tapping the corresponding marker line on the touchscreen in order to take over the current relative displacement and perform the corresponding settings, in particular with respect to the patient positioning. It is also conceivable for special operator elements for confirmation to be displayed on the touchscreen and/or additionally to the touchscreen, to be arranged, for example, adjacent thereto.

In another embodiment of the invention, following a displacement, such as in the case of the additional use of an operator confirmation action, a repeat acquisition of the at least one slice image to the position of which the marker line is assigned is performed with the new patient position and/or slice position. This immediately shows the user the consequences of the user's setting because the user is immediately shown the anatomy resulting from the changed positioning since an updated acquisition of the slice images is performed with changed patient positioning and/or in accordance with the change to the localization slice position, changed with respect to the target slices to be acquired. In the aforementioned example, therefore, a user displaces the marker line in the coronal slice image, such as following an operator confirmation action, in order to reposition the patient in the longitudinal direction, for example to displace a patient bed as the patient support in the longitudinal direction of the patient receiving area. Since, at the new patient position, there is also a new acquisition of the slice images, at least of the transverse slice image to which the marker line was assigned, the display of the new transverse slice image at the same time also shows the user the exact anatomy in this positioning. A similar procedure can be used in this example when a new position of the marker line assigned to the transverse slice image in the coronal slice image was selected. If no corresponding adjustability of the patient support apparatus was provided, this causes the positioning of the target slice to be acquired to change. Upon this change, and following an operator confirmation action, a new acquisition of the transverse slice image can be initiated in order to generate a preview for the target slice from which the user can determine whether the setting corresponds to the user's wishes.

This means that, in the example of a transverse and a coronal slice image, the transverse slice image can also be used in order to set the exact slice position of the coronal localization slice (and hence the target slice). Since the optimum slice position can be at different heights depending upon the patient's anatomy, moving the marker line in the transverse slice image enables another coronal localization slice or target slice to be selected, which can also be acquired once again.

In a further embodiment of the invention, at least two of the at least two slice images are displayed simultaneously. In this case, it is possible to use one single touchscreen in order to display multiple slice images adjacently, with correspondingly superimposed marker lines in order to improve the orientation of the user. For this purpose, the user is able to view multiple images, in particular all of the slice images, simultaneously, and also relate them to one another from the position of the marker lines. It is particularly expedient, in the case of a relative, still unconfirmed, displacement of the marker line and slice image with respect to one another by user interaction, for the previous position of the marker line for the slice image to continue to be displayed. This display of the marker line can be, for example, dashed and/or in another color, so that it remains available as an aid to orientation, in particular when the slice image to which the marker line was assigned was not updated by a new acquisition. In this context, reference is also made to the fact that it is generally advantageous, during the updating of slice images in response to user interactions, to store slice images that have already been acquired in order to avoid a repeat acquisition process on the repeat acquisition of a position that has already been visited.

As noted, particularly following an operator confirmation action, it is possible for the patient position to be set directly in that the position of the patient support apparatus is changed correspondingly. Specifically, an adjusting apparatus of the patient support is controlled when the patient position is set.

In a further embodiment of the invention, during the performance of a zoom operator action by interaction with the slice image displayed, a zoom function is performed for the magnified depiction of a section of the slice image selected during the zoom operator action. This zoom operator action can be achieved via the touchscreen, such as with a multi-touch operation to a defined area to be zoomed (two corners of a rectangle) and/or a zoom gesture. Such a gesture can be as used with everyday devices such as smartphones and/or tablets, by moving two fingers in the opposite direction from each other in order to zoom in on the slice image displayed. Therefore, if the depiction of the anatomy is not large enough for the user, the zoom operator action can be used to zoom in on the slice image.

In a further embodiment of the method, that an operator element is used for the repeat acquisition of at least one of the at least one slice image with higher resolution. Therefore, if a user considers the current resolution of slice images to be insufficient for the performance of precise positioning, a new measurement can be initiated in which at least one current slice image, preferably all slice images, are measured again with a higher resolution.

As noted, it is expedient for at least two of the at least two slice images to be displayed simultaneously. In this context, it is also conceivable for more than one touchscreen to be used, i.e. each slice image can be displayed on its own touchscreen. Other variants are also conceivable in which only a single touchscreen with a correspondingly adapted design is used on which at least two of the at least two slice images can be displayed simultaneously.

In addition to the method, the invention concerns a magnetic resonance apparatus having at least one touchscreen and a control computer designed to perform the method according to the invention. All explanations with respect to the method according to the invention apply analogously to the control computer according to the invention, with which the aforementioned advantages can be achieved. In particular, the control computer evaluates the operator signals detected by the touchscreen in order to identify user interactions or operator actions and carry out corresponding measures such as those described above with respect to the method according to the invention.

The magnetic resonance apparatus can be designed with the touchscreen arranged on the scanner of the magnetic resonance apparatus, outside the patient receiving area, in particular on a front side of the scanner. When the at least one touchscreen is provided on the end face of the magnetic resonance scanner from where the patient is introduced into the patient receiving area, the user has no problem in interacting with the touchscreen from there and carrying out the more precise positioning.

### BRIEF DESCRIPTION OF THE DRAWINGS

FIG. 1 shows a magnetic resonance apparatus according to the invention.

FIG. 2 depicts a coronal slice image on a touchscreen.

FIG. 3 depicts a transverse slice image on a touchscreen.

### DESCRIPTION OF THE PREFERRED EMBODIMENTS

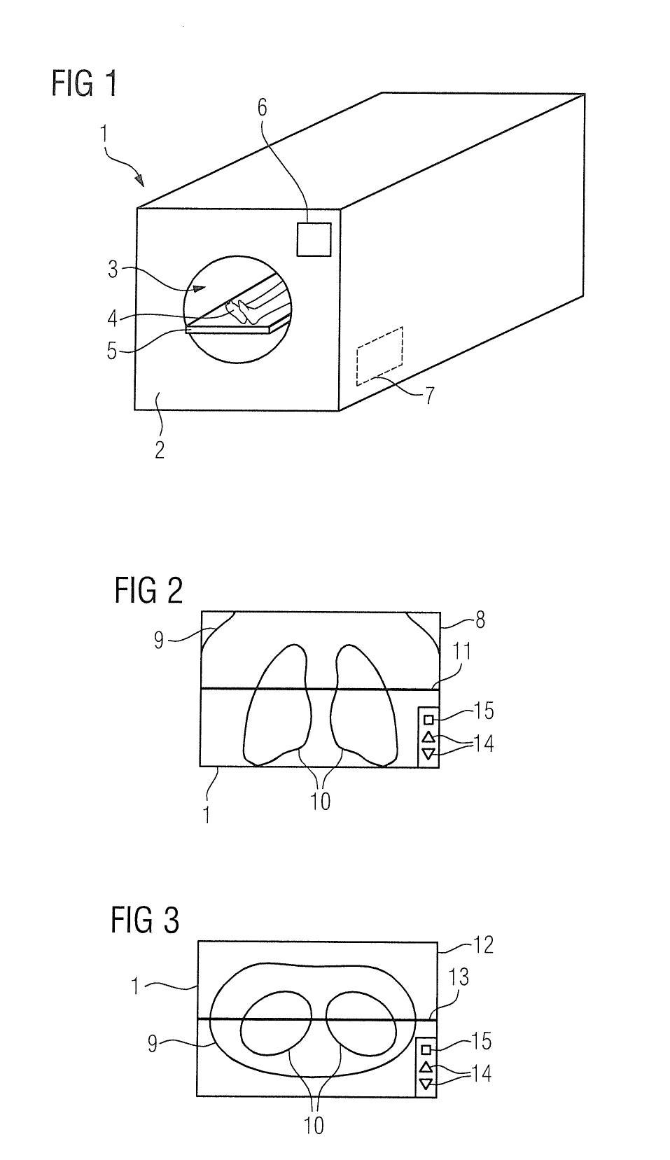

FIG. 1 is a schematic illustration of a magnetic resonance apparatus according to the invention 1. As is known in principle, the apparatus has a scanner 2 defining a patient receiving area 3. The patient receiving area 3 is surrounded, in a way not shown in further detail and as known in principle, by a radio-frequency coil arrangement and a gradient coil arrangement. A patient 4 can be introduced into the patient receiving area 3 by a patient support, here embodied as a patient bed 5. In the exemplary embodiment shown, the patient 4 is to be positioned more precisely, but was first placed in a starting position in the patient receiving area 3 that permits a first coarse positioning and was determined in dependence on the local coils connected (not shown in further detail here) and/or for example from patient parameters and/or examination parameters known from a radiology information system, wherein these parameters indicate which at least one anatomical structure is to be examined.

For precise positioning for the subsequent acquisition of magnetic resonance data in target slices to be acquired, the magnetic resonance apparatus 1 has a touchscreen 6 that can be operated by a user provided on the front side of the scanner 2 from where the patient 4 was also introduced in the patient receiving area 3.

The magnetic resonance apparatus 1 has a control computer 7 designed to carry out the method according to the invention, which will now be explained in more detail below.

When the patient 4 has been brought into described starting position by introducing the patient bed 5 into the patient receiving area 3, in the present exemplary embodiment, a coronal slice image lying in a coronal localization plane and a transverse slice image correspondingly lying in a transverse localization plane are acquired as localizers. With this initial acquisition of slice images, the localization slices pass through the center of the homogeneity volume, i.e. the isocenter of the magnetic resonance scanner 2. These two slice images are displayed adjacently on the touchscreen 6 such that the user can interact with them, and the maximum possible part of the display area is used. In the present case, only one touchscreen 6 is shown for use for the depiction of the two slice images, but it is also possible to use multiple touchscreens. Moreover, embodiments are also conceivable in which sagittal slice images are acquired additionally or alternatively.

FIG. 2 is a schematic depiction of a coronal slice image 8 such that is shown on the touchscreen 6. To simplify the depiction, only the outer contours 9 of the patient and the pulmonary lobes 10 are indicated. A marker line 11 indicated through the coronal slice image 8 and superimposed thereon in the depiction shows the relative position of the transverse slice image 12, which is depicted schematically in FIG. 3. Correspondingly, a marker line 13 indicating the position of the coronal localization slice, i.e. of the coronal slice image 8, is depicted superimposed on the transverse slice image 12.

FIGS. 2 and FIG. 3 both also show operator elements 14 for fine adjustment as arrows; a respective operator element 15 can be used as a confirmation operator element. Expediently, further operator elements are also provided on the touchscreen 6 or adjacent thereto, which are not shown here for the sake of clarity and are used to initiate a repeat acquisition of the slice images 8, 12 with higher resolution.

By touching the marker lines 11, 13, i.e. by user interaction therewith, the marker lines 11, 13 on the touchscreen can be displaced relative to the respective images 8, 12. Displacement of the marker line 11 enables the user to influence the patient positioning. If the marker line 11 is displaced and the displacement confirmed by an operator confirmation action, for example by double tapping the marker line 11 or actuating the operator elements 15, the patient bed 5 is moved automatically in accordance with the displacement described by displacement data, following which a repeat acquisition of at least the transverse slice image 12 is performed. The user thus is immediately informed which anatomy can now be viewed transversely with the new patient position, so that the user is able to check the previous action. Also conceivable is an embodiment in which, at the same time as the displacement of the marker line 11, it is always possible to acquire and display a new transverse slice image 12 at a position corresponding to the displaced marker line 11. Slice images 8, 12 that have already been acquired at specific positions are stored so that they do not have to be acquired several times. In this way, the user is finally able to “scroll” through the anatomy and thus find the anatomical structures which he or she wishes to acquire. In this case, the operator elements 14 are used for fine positioning, wherein, for example after the actuation of one of the arrows, the marker line 11 can be displaced by one single pixel only.

If the user wishes a magnified display of the anatomy, the user can perform a zoom operator action, for example by selecting a rectangular area to be zoomed with two fingers and/or by using a zoom gesture during which two fingers are moved apart in opposite directions from one another. In the case of a zoom gesture, the reverse gesture can be used to zoom back out again.

In the present case, manipulation of the marker line 13 does not result in changed patient positioning but instead changes acquisition parameters of a target slice to be acquired later with respect to its position. If the marker line 11 is displaced, optionally following an operator confirmation action, it is possible, in accordance with the new position of the marker line 13, for a new coronal slice image 8 to be acquired at this position in order to visualize the consequence once again. Also conceivable are exemplary embodiments in which the patient bed 5 is also vertically adjustable and therefore patient positioning can result from a user interaction with the marker line 13. The statements already made with respect to manipulation of the marker line 11 also apply with respect to the zoom function and the other operator elements 14, 15.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for setting a position in a magnetic resonance scanner that has a patient receptacle with a basic magnetic field homogeneity volume in the patient receptacle, said position being selected from the group consisting of a patient position of a patient in the patient receptacle and a slice position of a target slice from which magnetic resonance data are to be acquired, said method comprising:
operating the magnetic resonance apparatus, with the patient situated in the patient receptacle, to acquire the localization data representing two localization slices within the patient, said two localization slices being perpendicular to each other and having a defined position with respect to the homogeneity volume;
providing the localization data to a computer in communication with a touch screen;
in said computer, executing an algorithm to cause one of said two localization slices to be displayed on the touch screen together with a marker line that shows a position of the other of the two localization slices;
by user interaction with said touch screen, displacing at least one of said marker line and said slice image on said display screen to produce a relative displacement; and
in said computer, executing an algorithm to determine said position from said relative displacement, and making an electronic signal designating said position available as an output from said computer.

2. A method as claimed in claim 1 comprising acquiring said at least two localization slices as slices that perceive through a center of said homogeneity volume.

3. A method as claimed in claim 1 comprising selecting said at least two slice images as being respective slices from the group consisting of a transverse slice, a coronal slice and a sagittal slice.

4. A method as claimed in claim 1 comprising executing said user interaction as a displacement of the marker line on the slice image or a displacement of the slice image toward the marker line.

5. A method as claimed in claim 4 comprising additionally displaying an operator element on said touch screen and using said operator element, by user interaction with said touch screen, to execute a precise adjustment of said displacement.

6. A method as claimed in claim 4 comprising displaying said marker element on said touch screen superimposed on the slice image on the touch screen.

7. A method as claimed in claim 1 comprising after emitting said designation of said position from said computer, automatically effecting a change in said position only following a manual confirmation entered into said computer.

8. A method as claimed in claim 7 comprising, from said computer, operating a patient support of the magnetic resonance scanner on which the patient is situated to cause said patient to be positioned corresponding to said position, and thereafter operating the magnetic resonance scanner to acquire a slice image at a position corresponding to a position of said marker line.

9. A method as claimed in claim 1 comprising simultaneously displaying said at least two slice images at said touch screen.

10. A method as claimed in claim 1 comprising operating a patient support of said magnetic resonance scanner using said position designated at said output of said computer.

11. A method as claimed in claim 1 comprising, in said computer by manual action via said touch screen, executing a zoom algorithm to perform an enlarged depiction of at least a portion of said slice image on said touch screen, and operating the magnetic resonance scanner to acquire an image of said at least one slice on said touch screen with a higher resolution corresponding to said zoom operation.

12. A method as claimed in claim 1 wherein said touch screen is a first touch screen, and providing a second touch screen in communication with said computer, and respectively displaying each of said at least two localization images at said first and second touch screens together with a respective marker line representing the other of said at least two localization images.

13. A magnetic resonance apparatus comprising:
a magnetic resonance scanner having a patient receptacle adapted to receive a patient therein, said patient receptacle having a homogeneity volume;
a control computer in communication with a touch screen;
said control computer being configured to operate the magnetic resonance apparatus, with the patient situated in the patient receptacle, to acquire the localization data representing two localization slices within the patient, said two localization slices being perpendicular to each other and having a defined position with respect to the homogeneity volume;
said localization data being provided to said control computer, and said control computer being configured to execute an algorithm to cause one of said two localization slices to be displayed on the touch screen together with a marker line that shows a position of the other of the two localization slices;
said touchscreen being configured, by user interaction with said touch screen, to displace at least one of said marker line and said slice image on said display screen to produce a relative displacement; and
said control computer being configured to execute an algorithm to determine said position from said relative displacement, and to make an electronic signal designating said position available as an output from said computer.

14. A magnetic resonance apparatus as claimed in claim 13 wherein said touch screen is situated on said magnetic resonance scanner.

15. A magnetic resonance apparatus as claimed in claim 14 wherein said magnetic resonance scanner has a front side at which said patient is introduced into said patient receptacle of the magnetic resonance scanner, and wherein said touch screen is situated on said front side of said magnetic resonance scanner.
